SEQUENCE LISTING (7 sequences)

SEQ ID NO: 1 - DNA, 912 bp, Aspergillus niger
  CDS (1)..(909)
atgctatcaacccacctcctcttcctcgccaccaccctcctcacatccctcttccatcccattgccgcccatgtcgccaagcgcagtggtagcctccaacaaatcaccgatttcggtgataaccccacaggtgtaggcatgtacatctacgtgcctaataacctggcctcgaatcctggtatcgtggttgcaatccactactgcaccggcactggccccggctactacagcaactccccctacgccaccctctccgagcaatacggcttcatcgtgatctacccgtctagcccgtactccggaggctgctgggacgtgagttcgcaggcaacgctgacacataatggaggtggaaacagtaactcgattgccaacatggtcacctggacgattagcgagtacggggcggatagcaagaaggtgtttgtgacgggttcgagttcgggggctatgatgacgaacgtaatggcagcaacctaccccgaactcttcgccgctggcaccgtctactccggcgtctcagccggctgcttctactcggacactaaccaagtggacggatggaattccacctgcgcacaaggagacgtcatcaccaccccggaacactgggctagtattgccgaggcaatgtatccagggtactcgggaagccggccaaagatgcagatctaccacggcagtgtggatacgacgctgtatccgcagaattattatgagacgtgcaagcaatgggctggagtgtttgggtacgattacagtgcaccggaatcgacggaggcgaatactccgcagacgaattatgagacgacgatttggggagataatctgcaggggatctttgcgacgggcgtgggtcatacggtgccaattcatggggataaggatatggagtggtttgggtttgcttga

SEQ ID NO: 2 - protein, 303 aa, Aspergillus niger
MLSTHLLFLATTLLTSLFHPIAAHVAKRSGSLQQITDFGDNPTGVGMYIYVPNNLASNPGIVVAIHYCTGTGPGYYSNSPYATLSEQYGFIVIYPSSPYSGGCWDVSSQATLTHNGGGNSNSIANMVTWTISEYGADSKKVFVTGSSSGAMMTNVMAATYPELFAAGTVYSGVSAGCFYSDTNQVDGWNSTCAQGDVITTPEHWASIAEAMYPGYSGSRPKMQIYHGSVDTTLYPQNYYETCKQWAGVFGYDYSAPESTEANTPQTNYETTIWGDNLQGIFATGVGHTVPIHGDKDMEWFGFA

SEQ ID NO: 3 - protein, 303 aa, Aspergillus ficuum
MLSTHLLFLATTLLTSLFHPIAAHVAKRSGSLQQITDFGDNPTGVGMYIYVPNNLASNPGIVVAIHYCTGTGPGYYSNSPYATLSEQYGFIVIYPSSPYSGGCWDVSSQATLTHNGGGNSNSIANMVTWTISEYGADSKKVYVTGSSSGAMMTNVMAATYPELFAAGTVYSGVSAGCFYSDTNQVDGWNSTCAQGDVITTPEHWASIAEAMYPGYSGSRPKMQIYHGSVDTTLYPQNYYETCKQWAGVFGYDYSAPESTEANTPQTNYETTIWGDNLQGIFATGVGHTVPIHGDKDMEWFGFA

SEQ ID NO: 4 - protein, 304 aa, Aspergillus kawachii
MLLSTHLLFVITTLVTSLLHPIAAHAVKRSGSLQQVTDFGDNPTNVGMYIYVPNNLASNPGIVVAIHYCTGTGPGYYGDSPYATLSEQYGFIVIYPSSPYSGGCWDVSSQATLTHNGGGNSNSIANMVTWTISKYGADSSKVFVTGSSSGAMMTNVMAATYPELFAAATVYSGVSAGCFYSNTNQVDGWNSTCAQGDVITTPEHWASIAEAMYSGYSGSRPRMQIYHGSIDTTLYPQNYYETCKQWAGVFGYDYSAPEKTEANTPQTNYETTIWGDSLQGIFATGVGHTVPIHGDKDMEWFGFA

SEQ ID NO: 5 - protein, 304 aa, Aspergillus awamori
MLLSTHLLFVITTLVTSLLHPIDGHAVKRSGSLQQVTDFGDNPTNVGMYIYVPNNLASNPGIVVAIHYCTGTGPGYYGDSPYATLSEQYGFIVIYPSSPYSGGCWDVSSQATLTHNGGGNSNSIANMVTWTISKYGADSSKVFVTGSSSGAMMTNVMAATYPELFAAATVYSGVSAGCFYSNTNQVDGWNSTCAQGDVITTPEHWASIAEAMYSGYSGSRPRMQIYHGSIDTTLYPQNYYETCKQWAGVFGYDYSAPEKTEANTPQTNYETTIWGDSLQGIFATGVGHTVPIHGDKDMEWFGFA

SEQ ID NO: 6 - DNA, 29 bp, Artificial Sequence
  PCR primer
caccatgctatcaacccacctcctctcgc

SEQ ID NO: 7 - DNA, 33 bp, Artificial Sequence
  PCR primer
tcaagcaaacccaaaccactccatatccttatc

(19) United States
(12) Patent Application Publication
(10) Pub. No.: US 2015/0033411 A1
(43) Pub. Date: Jan. 29, 2015

(54) GENETICALLY MODIFIED PLANTS HAVING IMPROVED SACCHARIFICATION PROPERTIES

(71) Applicant: Swe Tree Technologies AB, Umeå (SE)

(72) Inventors: Prashant Mohan Pawar, Umea (SE); Marta Derba-Maceluch, Umea (SE); Ewa J. Mellerowicz, Umeå (SE); Madhavi Latha Gandla, Kurnool (IN); Leif Jönsson, Umea (SE)

(21) Appl. No.: 14/384,649

(22) PCT Filed: Mar. 12, 2013
(86) PCT No.: PCT/SE2013/050226

§ 371 Date: Sep. 11, 2014

Related U.S. Application Data
(60) Provisional application No. 61/610,963, filed on Mar. 14, 2012.

(30) Foreign Application Priority Data
Mar. 14, 2012 (SE) 1250240-7

(51) Int. Cl. C12N 15/82

(52) U.S. Cl. CPC C12N 15/8205; C12N 15/8245
USPC 800/279; 800/284; 800/298

(57) ABSTRACT

The invention relates to methods for increasing saccharification potential in a plant, comprising overexpressing a polynucleotide encoding an acetyl xylan esterase polypeptide in at least one cell type in said plant. The invention further relates to methods for producing genetically modified plants overexpressing a polynucleotide encoding an acetyl xylan esterase polypeptide, as well as to genetically modified plants produced by such methods.

### TECHNICAL FIELD

The invention relates to methods for increasing saccharification potential in a plant, comprising overexpressing a polynucleotide encoding an acetyl xylan esterase polypeptide in at least one cell type in said plant. The invention further relates to methods for producing genetically modified plants overexpressing a polynucleotide encoding an acetyl xylan esterase polypeptide, as well as to genetically modified plants produced by such methods.

### BACKGROUND ART

Xylan is one of the main compounds of lignocellulose and constitutes a large part of usable biomass for human exploitation. The hardwood xylan from various species and the xylan of forage crops is usually heavily acetylated. The presence of acetyl groups affects many properties of lignocellulose such as cross-linking and extractability and reactivity. Moreover, xylan hydrolysis to obtain xylose, is heavily hampered by the presence of acetyl groups on xylan backbone, necessitating either enzymatic or chemical treatment prior acetyl removal, leading to high costs and/or environmental hazards.

Xylan is the third most abundant biopolymer found on earth and it contributes to large amount of biomass available for human exploitation. Xylan backbone consists of β-(1→4) linked D-xylopyranosyl residues substituted with 4-O-methyl-D-glucuronic acid/glucuronic acid. The xylopyranosyl residues are partially acetylated in the C-2 and/or C-3 positions. Xylan acetylation might affect the conversion of lignocellulosic biomass to fermentable sugar, which is a crucial step in biofuel production, and it might affect the microorganisms fermenting sugars to ethanol. It also might be important for xylan cell wall physico-chemical properties. Total acetyl content in aspen wood is about 3%-5% and most of it is associated with xylan. Acetyl content in wheat straw, bagasse and switch grass is about 2%-3%.

Decrease in acetyl content by chemical pretreatment improves the sugar yield. In a study of P. tremula, deacetylation of wood by KOH treatment increased sugar yield from 12% to 42%. In a similar study of P. tremuloides, reduction of acetyl content by 85% of its original value resulted in the doubling of glucan conversion and in 8 times higher xylan conversion. Moreover, when the lignocellulose is de-acetylated, milder delignification treatment could be applied for effective saccharification. Similar observations were made in the case of straw of grasses and cereals. The presence of acetyl groups in lignocellulose is a disadvantage for biofuel production not only during saccharification but also during subsequent fermentation. Too high concentration of acetic acid inhibits microbial fermentation.

Wood deacetylation plays an important role in the chemo-thermo-mechanical pulping. It favorably changes the architecture of cell wall increasing fiber swelling and effective capillarity of fibers. The deacetylation substantially reduces solubility of hemicelluloses and increases their adsorption onto cellulose fibers, which improves bonding capacity of the fibers and increases their yield.

Thus, acetyl needs to be removed from lignocellulose in these applications. A common strategy to remove acetyl is the pretreatment with bases. It has been shown that 100 g of wood require approx 4 g of KOH for complete deacetylation. Although the dilute base pretreatment would remove acetate specifically without affecting xylan or lignin, this will increase the overall production costs. For example, according to current estimations, 20% difference in the lignocellulose acetylation translates to 10% difference in the price of ethanol.

Arabidopsis plants with 40% lower than WT acetyl content of xylan were obtained by mutating RWA genes involved in polysaccharide acetylation (Lee et al. (2011) Plant and Cell Physiology 52: 1289-1301). This reduction did not lead to increased cellulose digestibility in saccharification without pretreatment.

Pogorelko et al. (2011) Plant Mol Biol 77:433-445, constructed an expression cassette composed of the Cauliflower Mosaic Virus 35 S RNA promoter, the Arabidosis thaliana β-expansin signal peptide, and the fluorescent marker protein YFP. The authors introduced into Colombia-0 plants three Aspergillus nidulans hydrolases, β-xylosidase/α-arabinosidase, feruloyl esterase, acetylxylan esterase (AnAXE), and a Xanthomonas oryzae putative a-L-arabinofuranosidase. Acetyl content in AnAXE plants was reduced by 23% in comparison with Col-0 plants. There was no increase in saccharification after acid pretreatment.

Fusion with YFP permitted quick and easy screening of transformants, detection of apoplastic localization, and protein size confirmation. Compared to wild-type Col-0, all transgenic lines showed a significant increase in the corresponding hydrolytic activity in the apoplast and changes in cell wall composition. Examination of hydrolytic activity in the transgenic plants also showed, for the first time, that the X. oryzae gene indeed encoded an enzyme with α-L:-arabinofuranosidase activity. None of the transgenic plants showed a visible phenotype; however, the induced compositional changes increased the degradability of biomass from plants expressing feruloyl esterase and β-xylosidase/α-arabinosidase. Our results demonstrate the viability of creating a set of transgenic A. thaliana plants with modified cell walls to use as a toolset for investigation of how cell wall composition contributes to recalcitrance and affects plant fitness.

There are indications that a too high deacetylation might induce recalcitrance by reducing polymer solubility (Poutanen et al. (1990) Appl Microbiol Biotechnol 33: 506-510).

The acetyl xylan esterase (axe A) gene from Aspergillus niger (SEQ ID NO: 1) has been disclosed with GenBank accession No. A22880.1 and NCBI Reference Sequence XM_{—}001395535.2. The corresponding polypeptide is shown as SEQ ID NO: 2. Acetyl xylan esterases from other species are known in the art. For instance, acetyl xylan esterases from the Aspergillus species ficuum, kawachii and awamori, are shown as SEQ ID NO: 3, 4 and 5, respectively.

There is a need for improved methods for Xylan deacetylation in plants, in order to improve extractability, reactivity, enzymatic digestibility, saccharification, and fermentation behavior.

### BRIEF DESCRIPTION OF THE DRAWINGS

FIG. 1. Transgenic Arabidopsis lines expressing xylan esterase (transgene). Actin expression (AT3G18780.2) shows uniform cDNA loadings for the WT (Col 0) and the transgenic lines. Note the normal growth of the transgenic lines. Line 6c was analyzed in duplicate, using seeds from 2 different plants.

FIG. 2. Xylan acetyl esterase activity in protein extracts from the stem tissues of transgenic lines and the WT. Means of total activity soluble (S) and wall bound (W) and SE of two biological replicates of pooled 10 stems per replicate. Statistically significant increase in total activity in transgenic lines compared to WT is indicated by stars: *=P≦10%; **=P≦5%, (t test). Y-axis: Specific activity in nmol/min/mg.

FIG. 3. Morphology and growth of transgenic plants.

Panel A: Dry weight in gram (Y-axis) of various plant parts. L+S; Leaves+stem; R+R Rosette+root.

Panel B: Water content (% Y-axis) of various plants parts. L+S; Leaves+stem; R+R Rosette+root.

The plant parts are (i) stem and leaves; and (ii) root and rosette. The values are mean values from 30 plants±standard error SE. Statistically significant change in transgenic lines compared to WT is indicated by stars: *=P≦10%; **=P≦5%, (t test).

FIG. 4. Suseptibility to biotrophic pathogen Hyaloperonospora arabidopsidis of transgenic line 6c and WT. The y-axis represents number of spores mg-1 fresh weight. Mean of 10 experiments±SE. The difference was significant at P≦0.0522 (ANOVA).

FIG. 5. Morphology of wood cell size and shape in transgenic lines over expressing fungal CE1 and the WT. Cell dimensions were determined in macerates made from hypocotyls of 5 plants per line pooled together. N=200 cells, bars=SE. Line 6c was analyzed in duplicate using seeds from 2 different plants. Statistically significant difference in transgenic lines compared to WT is indicated by stars: *=P≦10%; **=P≦5%, (Student t test). FIG. 5A is representing fiber length, FIG. 5B fiber width, FIG. 5C vessel element length, and FIG. 5D vessel width, respectively.

FIG. 6. FT-IR analysis of Arabidopsis lines expressing xylan esterase and the WT.

Panel A: OPLS-DA analysis showing separation of transgenic lines and the WT.

Panel B: Loadings plots showing spectra contributing to the separation. Spectra associated with acetate and adsorbed water are shown. Analysis indicates more acetate and less adsorbed water in the WT. Data points are spectra of stem ground powder from 9 plants.

FIG. 7. Cell wall acetyl content. Analysis was performed on stem powder using 3 biological replicates in each transgenic line and 5 biological replicates in the WT. Bars=SE. Statistically significant decrease in acetyl content in transgenic lines compared to WT is indicated by stars: *=P≦10%; **=P≦5%, (Student t test). Y-axis is representing the content of acetic acid in mg/g.

FIG. 8. MALDI-AP analysis of neutral xylo-oligosaccharides obtained by xylanase digestion of cell wall preparations of transgenic lines and WT.

Panel A: Increased accessibility of xylan in transgenic lines is indicated by the lower content or lack of xylotetraose (xyl4) in xylanase digest. Y-axis is representing the xylo-oligosaccharides signal, Intensities distribution in %.

Panel B: Oligosaccharides containing acetyl group(s) were identified. Acetylation index was calculated as a percentage of intensities of acetylated oligosaccharides having a defined number of acetyl groups per xylose multiplied by DA of a given oligosaccharide, in total signal. This indicated lower relative content of acetylated xylo-oligasaccharides in the transgenic lines compared to WT. Means of 3 biological replicates and SE are shown. Each biological replicate consisted of 3 plants.

FIG. 9. MALDI-TOF analysis of xylogluco-oligosaccharides released by xyloglucanase digestion of cell wall preparations of transgenic lines and the WT. Values represent relative content. The content of acetylated oligos containing galactose (FIG. 9A) was reduced in the line 6c compared to WT, whereas the content of non-acetylated (FIG. 9B) such oligos was increased. N=6, bars=SE

FIG. 10. Saccharification rates in the transgenic Arabidopsis lines and WT without pretreatment (A), with alkali pretreatment (B) and with acid pretreatment (C). Data with percentages shown correspond to individual lines significantly different from WT at P≦5% (Student t-test). Significance of the contrast of all transgenic lines versus WT is shown above the bars. Each line was represented by 30 plants. Means of 4 technical replicates and SE.

FIG. 11. Relative carbohydrate (A) and relative lignin (B) contents determined by pyrolysis-GC in transgenic Arabidopsis lines expressing xylan esterase and the WT. Means+/−SE of three biological replicates for the transgenic lines or six biological replicates for the WT. Each biological replicate consisted of 3 plants. S-lignin (S), G-lignin (G) and H-lignin (H). Line 6c was analyzed in duplicate using seeds from 2 different plants. Differences among lines were not statistically significant by ANOVA (P<10%). AIR2 is de-starched alcohol insoluble residue.

FIG. 12. Updegraff cellulose (A) and Klason lignin (B) contents in transgenic Arabidopsis lines expressing xylan esterase and the WT. AIR1—alcohol insoluble residue; AIR2—de-starched alcohol insoluble residues. Means+/−SE of three biological replicates for the transgenic lines or six biological replicates for the WT. Each biological replicate consisted of 10 plants. Differences among lines were not statistically significant by ANOVA (P<10%).

FIG. 13. Presence of transgene transcript in aspen lines carrying 35S::CE1 construct and in the WT detected by RT-PCR of in vitro grown stem tissues. Ubiquitin transcript is shown as a reference for loading.

FIG. 14. Esterase activity in developing wood of transgenic aspen lines and WT aspen measured using p-Napthyl Acetate as the substrate. Means of total activity (soluble, S and wall-bound, W) of six trees per transgenic line and WT, bars=SE. Statistically significant increase in activity in transgenic lines compared to WT is indicated by stars: *=P≦10%; **=P≦5%, (Student t test). The Y-axis is representing the specific activity in nmol/min/mg.

FIG. 15. Height (Fig. A) and diameter (Fig. B) growth in the greenhouse of transgenic aspen lines and the WT. Bars=SEs. Means of 10 plants per transgenic line and 21 plants per WT. Lines 5, 8, and 11 were significantly taller, and line 4 was significantly shorter than the WT (Student t test, P≦5%). No significant differences in the internode diameter were observed.

FIG. 16. Total cell wall acetyl content in the wood of transgenic lines and WT aspen. Means six biological replicates per transgenic line and 10 biological replicates per WT. All lines showed significant decrease in acetyl content compared to WT (Student t test), down to 85% of WT level in line 4.

FIG. 17. MALDI-AP analysis of neutral xylo-oligosaccharides obtained by xylanase digestion of cell wall preparations of wood of transgenic lines and WT aspen. A. Increased accessibility of xylan in transgenic lines compared to WT is indicated by the lower content of xylotetraose and higher content of xylobiose in xylanase digest.

B. Neutral oligosaccharides containing acetyl group(s) were identified. Acetylation index was calculated as a percentage of intensities of acetylated oligosaccharides having a defined number of acetyl groups per xylose multiplied by DA of a given oligosaccharide in total signal. This indicated lower relative content of acetylated xylo-oligasaccharides in the transgenic lines compared to WT. Means of 2 biological replicates and SD.

FIG. 18. FTIR analysis of wood in the transgenic lines and WT aspen. Loading plot showing spectra discriminating between the transgenic lines and the WT. Note that the discriminating spectra included the signals from acetyl groups at 1240, 1370 and 1740 cm-1, showing decrease and the signals from 1659 cm-1, showing increase in the transgenic lines compared to the WT.

FIG. 19. Relative carbohydrates (A) and relative lignin (B) contents determined by pyrolysis-GC in transgenic aspen lines expressing xylan esterase and the WT. Means SE of eight biological replicates for the transgenic lines or 21 biological replicates for the WT. Differences among lines were not statistically significant for carbohydrate content by ANOVA (P<10%). S and G lignin content was decreased or increased in line 4, respectively compared to WT at P≦5% (Student t-test) and unchanged in other lines. H=H-lignin

FIG. 20. Sugar yields, determined by using ion chromatography, for transgenic 35S::CE1 aspen lines and WT aspen after acid pretreatment. Sugar yield: g of each monosaccharide per g of wood after 72 h of hydrolysis. Error bars show standard deviations. Lines 4, 5, 8, 11, 17: average of 5 pooled samples. WT: average of 5 samples. Data with percentages shown correspond to individual lines that differ significantly from the WT at P≦5% (Student's t-test). The P value for all transgenic lines combined versus the WT is shown above the bars.

FIG. 20A represents sugar yields in pretreatment liquids. The yields of arabinose and galactose were <0.01 g/g. On average, the yield of glucose for the transgenic lines was 75% higher than that of the WT. The yields of mannose in the hydrolysates of lines 11, 17 and WT were <0.01 g/g. The mannose yields of lines 4, 5 and 8 were significantly higher (P<0.05) than that of the WT.

FIG. 20B represents sugar yields in hydrolysates. The yields of arabinose, galactose and xylose were <0.01 g/g. On average, the yield of glucose of the transgenic lines was 10% higher than that of the WT.

FIG. 21. Total sugar yield (after pretreatment and enzymatic hydrolysis), for transgenic 35S::CE1 aspen lines and WT aspen after acid pretreatment. Sugar yield: g of each monosaccharide per g of wood after 72 h of hydrolysis. Error bars show standard deviations. Lines 4, 5, 8, 11, 17: average of 5 pooled samples. WT: average of 5 samples. Data with percentages shown correspond to individual lines that differ significantly from the WT at P≦5% (Student's t-test). The P values of all transgenic lines combined versus the WT are shown above the bars. Fig. A represents the total yield of each monosaccharide. The yields of arabinose and galactose were <0.01 g/g. On average, the yield of glucose of the transgenic lines was 13% higher than that of the WT. The yield of mannose in the hydrolysates of lines 11, 17 and WT was <0.01 g/g. The mannose yields of lines 4, 5 and 8 were significantly higher (P<0.05) than that of the WT. Fig. B represents the total sugar yields in the form of hexoses and pentoses. The values for hexoses indicate the total yields of galactose, glucose and mannose. The values for pentoses indicate the total yields of arabinose and xylose. On average, the yields of hexoses for the transgenic lines were 14% higher than that of the WT, while the pentose yields of the transgenic lines were almost equal to that of the WT.

FIG. 22. Yield of acetic acid (g/g) from transgenic 35S::CE1 aspen lines and WT aspen. Yield of acetic acid: g of acetic acid per g of wood after 72 h of hydrolysis. Error bars show standard deviations. Lines 4, 5, 8, 11, 17: average of 5 pooled samples. WT: average of 5 samples. Data with percentages shown correspond to individual lines that differ significantly from that of the WT at P≦5% (Student's t-test). The P value of all transgenic lines combined versus the WT is shown above the bars. Fig. A shows acetic acid yield in the pretreatment liquid after acid pretreatment. On average, the yield of acetic acid in the lines was 4% lower than that of the WT. The yield from line 4 was 13% lower than that of the WT. Fig. B shows acetic acid yield in the hydrolysates without pretreatment. On average, the yield of acetic acid from the transgenic lines was 4% lower than that of the WT. The yield of line 4 was 11% lower than that of the WT.

### DISCLOSURE OF THE INVENTION

The inventors have used a fungal (Aspergillus niger) xylan esterase gene to express xylan esterase activity in plant cell walls. It has surprisingly been shown that overexpression of acetyl xylan esterase decreases lignocellulose acetylation in the transgenic plants, without compromising their growth and cellulose content, and that higher saccharification yields are obtained from the transgenic plants as compared to the wild type not only in saccharification without a pretreatment, but also when alkali and acid pretreatments were applied. Therefore the transgenic plants are useful as bioenergy crops or in the development of bioenergy crops. In addition, a better fiber pulping is expected.

Unexpectedly the present invention shows that in the case of herbaceous plant (Arabidopsis) a reduced deacetylation of about 12% (between 0-34%) according to an unmodified plant of the same type will improve the saccharification without chemical pretreatment and the saccharification with alkali pretreatment, with no recalcitrance in the plant. This is shown in FIGS. 7 and 10, and summarized in Table 1. Moreover, the present invention also shows that in the case of woody plant (Aspen) a reduction in acetylation of about 13% (between 11-16%) as compared with unmodified woody plant, improved the saccharification without pretreatment and with acid pretreatment. This is demonstrated in FIGS. 16, 20 and 22, and summarized in Table 2.

Consequently, in a first aspect the invention provides a method of increasing saccharification potential in a plant, said method comprising overexpressing a polynucleotide encoding an acetyl xylan esterase polypeptide in at least one cell type in said plant.

The term “saccharification” means the process of converting complex carbohydrate or polysaccharides into simple monosaccharide components (e.g. glucose) through hydrolysis. The term “saccharification potential” means the amount of monosaccharides that can be released from the polysaccharides. In particular, the methods of the invention are useful for improving glucose yields in plants.

In a further aspect, the invention provides a method for producing a genetically modified plant, said method comprising overexpressing a polynucleotide encoding an acetyl xylan esterase polypeptide in at least one cell type in said plant. According to the invention the said plant has increased saccharification as compared to a corresponding non-genetically modified wild-type plant.

Preferably, the said methods comprise transforming said cell type with an expression cassette comprising a promoter that is functional in a plant cell, said promoter being operably linked to a polynucleotide encoding an acetyl xylan esterase polypeptide, and said promoter regulating overexpression.

The said promoter is preferably a CaMV 35S promoter, an ectopically expressing promoter such as the ubiquitin promoter, or any type of promoter expressing in cells with secondary cell walls, such as 4CL1.

In a preferred form of the invention, the said polynucleotide has a nucleotide sequence identical with SEQ ID NO: 1 of the Sequence Listing. However, the polynucleotide is not to be limited strictly to the sequence shown as SEQ ID NO: 1. Rather the invention encompasses polynucleotides carrying modifications like substitutions, small deletions, insertions or inversions, which nevertheless encode proteins having substantially the biochemical activity of the acetyl xylan esterase polypeptide according to the invention. For instance, the polynucleotide can be at least 60%, 70%, 80%, 90%, or 95% homologous with the nucleotide sequence shown as SEQ ID NO: 1 in the Sequence Listing.

Consequently, in the methods according to the invention the said polynucleotide is preferably selected from:

(a) polynucleotides comprising the nucleotide sequence of SEQ ID NO: 1;
(b) polynucleotides comprising a nucleotide sequence capable of hybridizing, under stringent hybridization conditions, to a nucleotide sequence complementary the polypeptide coding region of a polynucleotide as defined in (a) and which codes for a biologically active acetyl xylan esterase polypeptide or a functionally equivalent modified form thereof; and
(c) polynucleotides comprising a nucleic acid sequence which is degenerate as a result of the genetic code to a nucleotide sequence as defined in (a) or (b) and which codes for a biologically active acetyl xylan esterase polypeptide or a functionally equivalent modified form thereof.

The term “stringent hybridization conditions” is known in the art from standard protocols and could be understood as e.g. hybridization to filter-bound DNA in 0.5 M NaHPO₄, 7% sodium dodecyl sulfate (SDS), 1 mM EDTA at +65° C., and washing in 0.1×SSC/0.1% SDS at +68° C.

The phrase “degenerate as a result of the genetic code” is well known in the art. A sequential grouping of three nucleotides (a codon) codes for one amino acid. Since there are 64 possible codons, but only 20 natural amino acids, most amino acids are coded for by more than one codon. This phenomenon is referred to as the natural “degeneracy”, or “redundancy”, of the genetic code. It will thus be appreciated that the nucleotide sequence shown in the Sequence Listing is only an example within a large but definite group of sequences which will encode the acetyl xylan esterase polypeptide.

In one embodiment of the methods according to the invention, the said acetyl xylan esterase polypeptide is selected from:

(a) polypeptides comprising the amino acid sequence shown as SEQ ID NO: 2, 3, 4, or 5;
(b) polypeptides consisting essentially of the amino acid sequence shown as SEQ ID NO: 2, 3, 4 or 5; and
(c) polypeptides consisting of the amino acid sequence shown as SEQ ID NO: 2.

However, it will be understood by the skilled person that acetyl xylan esterases from other species than Aspergillus will also be useful in methods according to the invention. For instance, the invention encompasses the use of polypeptides carrying modifications like substitutions, small deletions, insertions or inversions, which polypeptides nevertheless have substantially the biological activities of acetyl xylan esterase. Included in the invention is consequently the use of polypeptides, the amino acid sequence of which is at least 60%, 70%, 80%, 85%, 90%, or 95% homologous, with the amino acid sequence shown as SEQ ID NO: 2, 3, 4, or 5 in the Sequence Listing.

The transgenic plant is preferably selected from angiosperms and other plants that possess acetylated xylan in cell walls, such as poplars, eucalypts, willows, and grasses.

Included are also acacia, hornbeam, beech, mahogany, walnut, oak, ash, hickory, birch, chestnut, alder, maple, sycamore, ginkgo, palm tree, sweet gum, cypress, Douglas fir, fir, sequoia, hemlock, cedar, juniper, larch, pine, redwood, spruce, yew, bamboo, switch grass, red canary grass, Miscantus species and rubber plants.

More preferably, the plant is from the Salicaceae family, e.g. from the Salix or Populus genera. Members of these genera are known by their common names: willow, poplar and aspen.

Included in the invention are methods wherein the plant or a part of the plant is pretreated with a suitable agent, such as acid or alkali, prior to enzymatic hydrolysis.

The invention further comprises genetically modified (transgenic) plants produced by the methods as described above. Specifically, the said genetically modified plant is overexpressing a polynucleotide encoding an acetyl xylan esterase polypeptide in at least one cell type in said plant. According to the invention, such plants have increased saccharification potential as compared to a corresponding non-genetically modified wild-type plant.

### EXAMPLES

### Example 1

### Transformation of Plants with the Acetyl Xylan Esterase Gene

cDNA (SEQ ID NO: 1) encoding Aspergillus niger acetyl xylan esterase was amplified using the following primers:

| (SEQ ID NO: 6) | |
|---|---|
| 5′ Fc2fuf | (caccATGCTATCAACCCACCTCCTCTCGC); |
| and | |
| (SEQ ID NO: 7) | |
| 3′ Fc2r1s | (TCAAGCAAACCCAAACCACTCCATATCCTTATC). |

The obtained PCR product was cloned into the pENTR™/D-TOPO® plasmid by using TOPO® Cloning System (Invitrogen, Carlsbad, Calif., USA K2400-20) and then transferred into pK2GW7 (Karimi, M. et al. (2002) Trends Plant Sci. 7(5): 193-195), using Gateway® Cloning System (Invitrogen, Carlsbad, Calif., USA). The resulting vector was transformed into Agrobacterium strain GV3101 (pMP90RK) by electroporation and colonies containing plasmid were selected on LB plates with following antibiotics: Rifampicin (10 μg/mL⁻¹), Gentamycin (30 mg/mL⁻¹), Kanamycin (30 μg/mL⁻¹) and Spectinomycin (50 μg/mL⁻¹). Agrobacterium-mediated transformation of Arabidopsis thaliana was performed as described by Clough and Bent (1998) Plant J 16:735-743. Transformed plants were selected on ½MS medium with 1% sucrose and kanamycin (50 m/mL⁻¹). Aspen plants were transformed by the same Agrobacterium strain using stem and petiole segments as known in the art.

### Example 2

### Effects of CE1 Expression in Arabidopsis

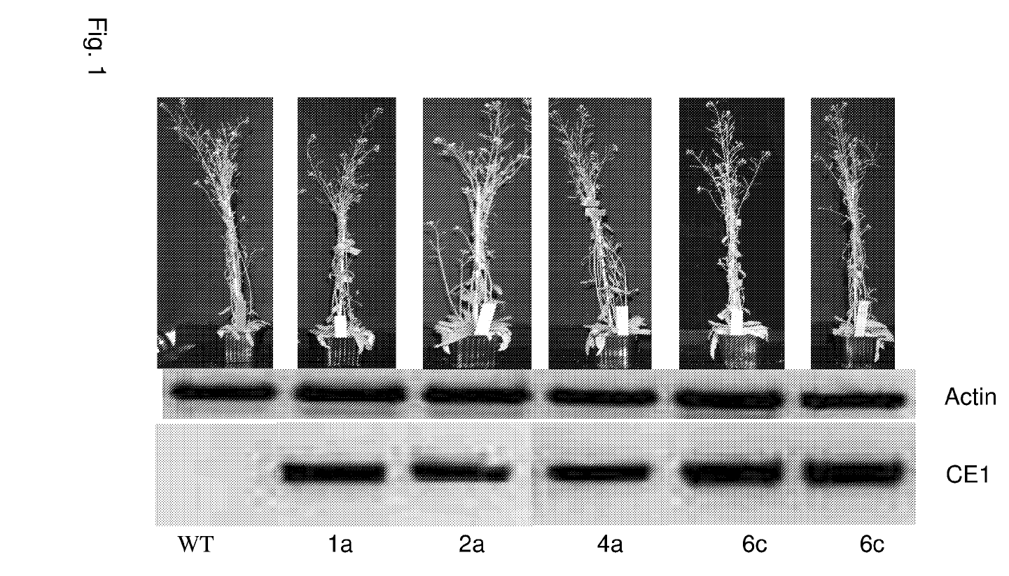

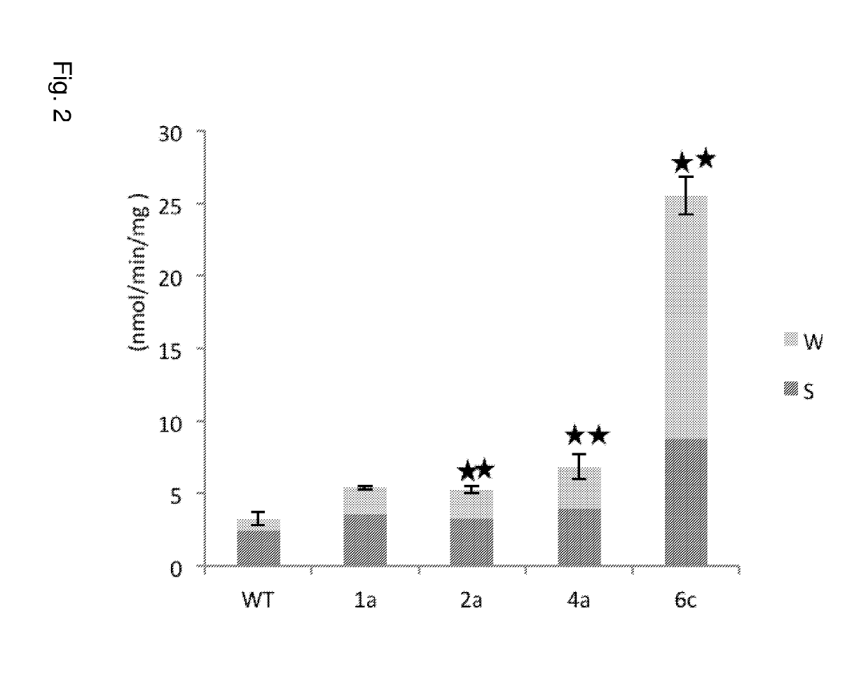

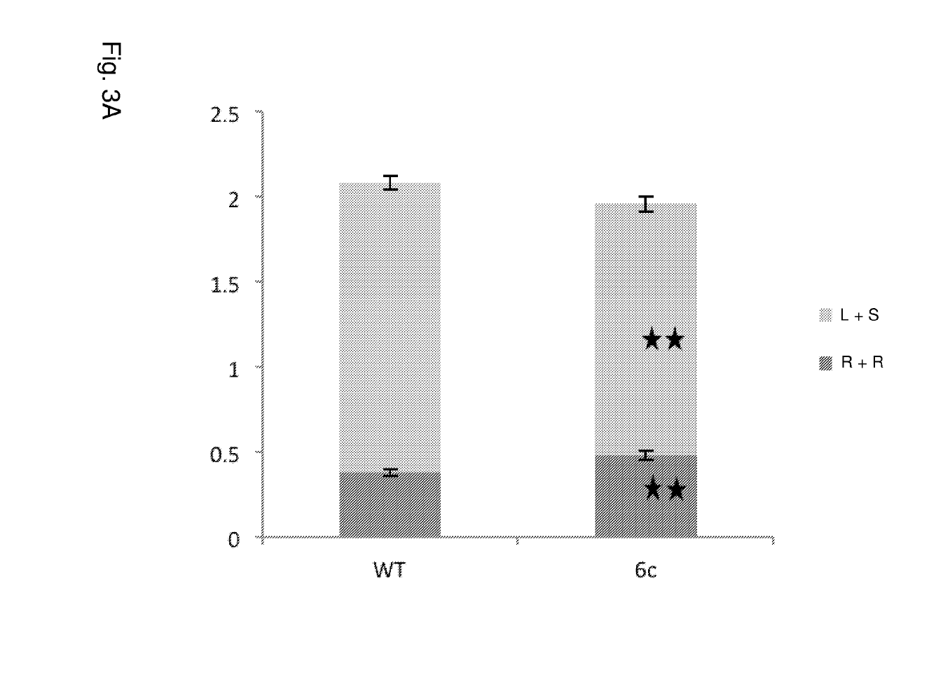

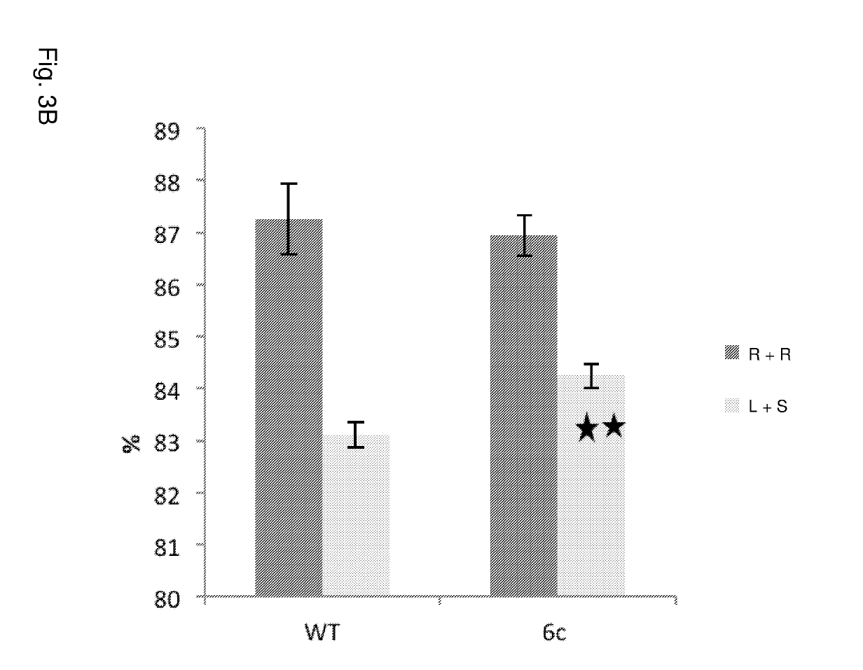

In Arabidopsis, four independent, single insert, homozygotic lines were analyzed. Expression of the transgene was detected by reverse transcription-polymerase chain reaction (RT-PCR). Line 6c had the highest transcript level (FIG. 1).

Transgenic lines grew normally till maturity (FIG. 1). Early seedling growth on plates (MS+sucrose) was not significantly affected.

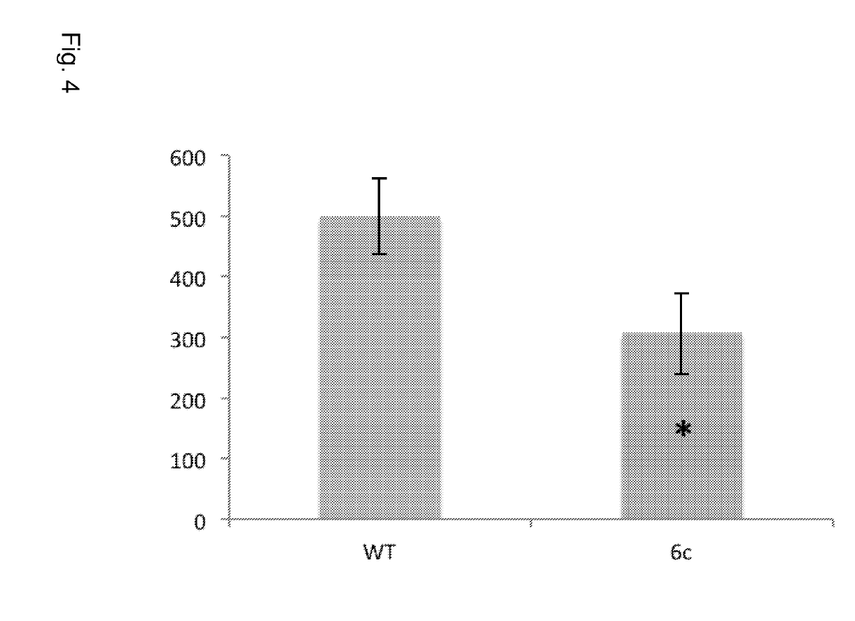

Xylan acetic esterase activity was determined in the transgenic lines using pNP substrate. Both soluble and wall-bound protein fractions of transgenic lines had a higher esterase activity compared to WT (FIG. 2).

The morphology and growth of the transgenic plants did not visibly differ from that of the WT plants. We measured the biomass of the most highly expressing line 6c. The biomass did not differ, but there was a small significant shift from the stem to rosette leaves and roots. The water content of the stem was slightly increased (FIG. 3).

To check if the introduced transgene caused increased susceptibility to biotic stresses, the susceptibility to a biotrophic pathogen of Arabidopsis, Hyaloperonospora arabidopsidis, was tested for the most highly expressing line 6c. The plants were exposed to the inoculum and the number of spores produced by the pathogen on the leaves of the plants was recorded. Transgenic plants exhibited a fewer number of spores than WT plants (FIG. 4), indicating their increased resistance to the pathogen.

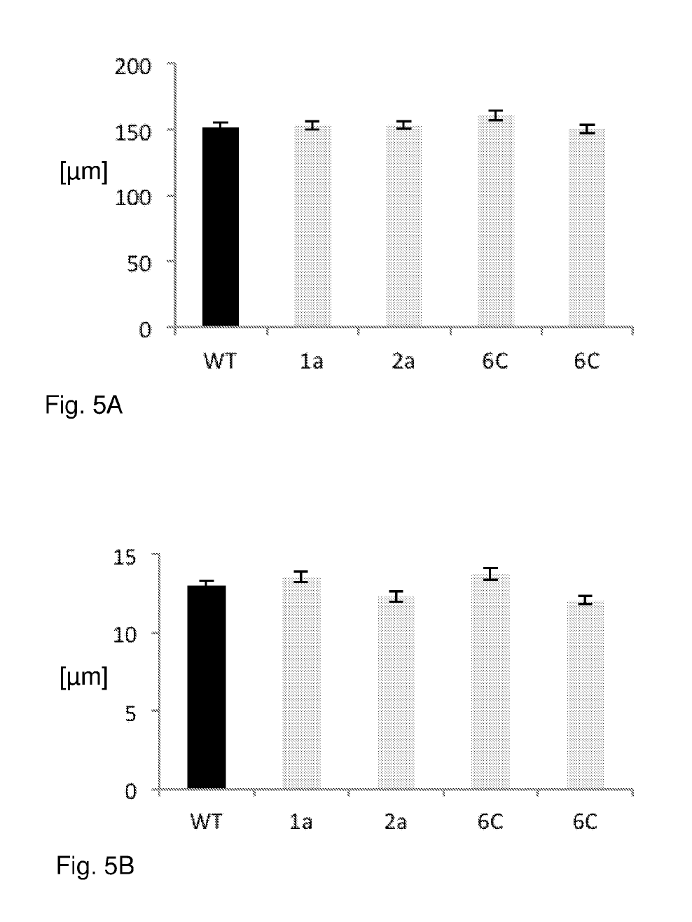

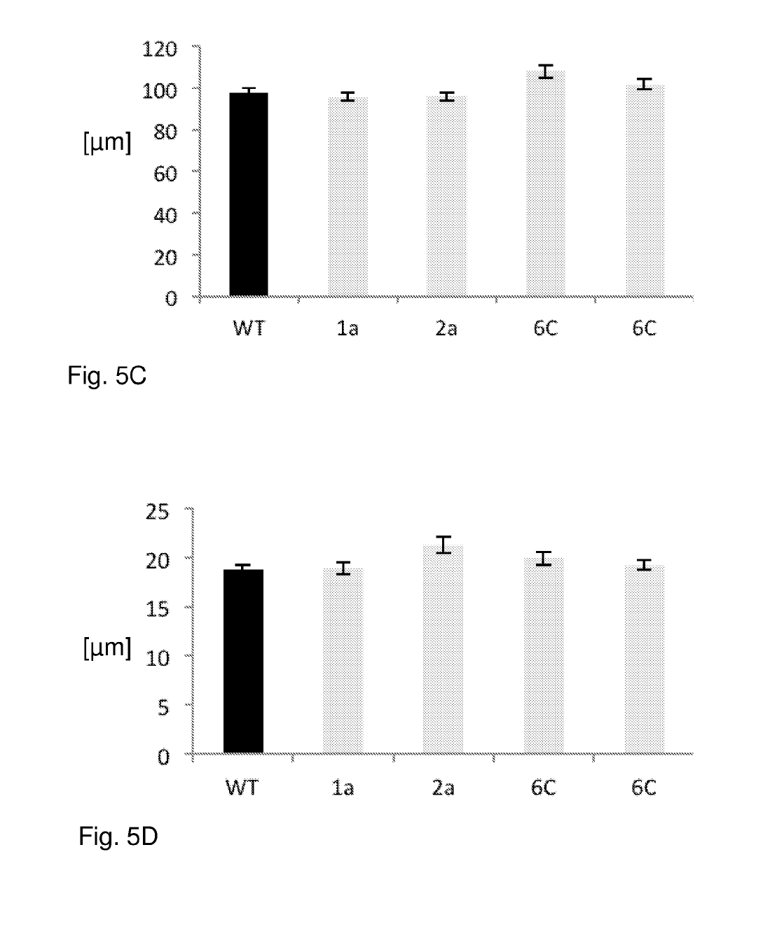

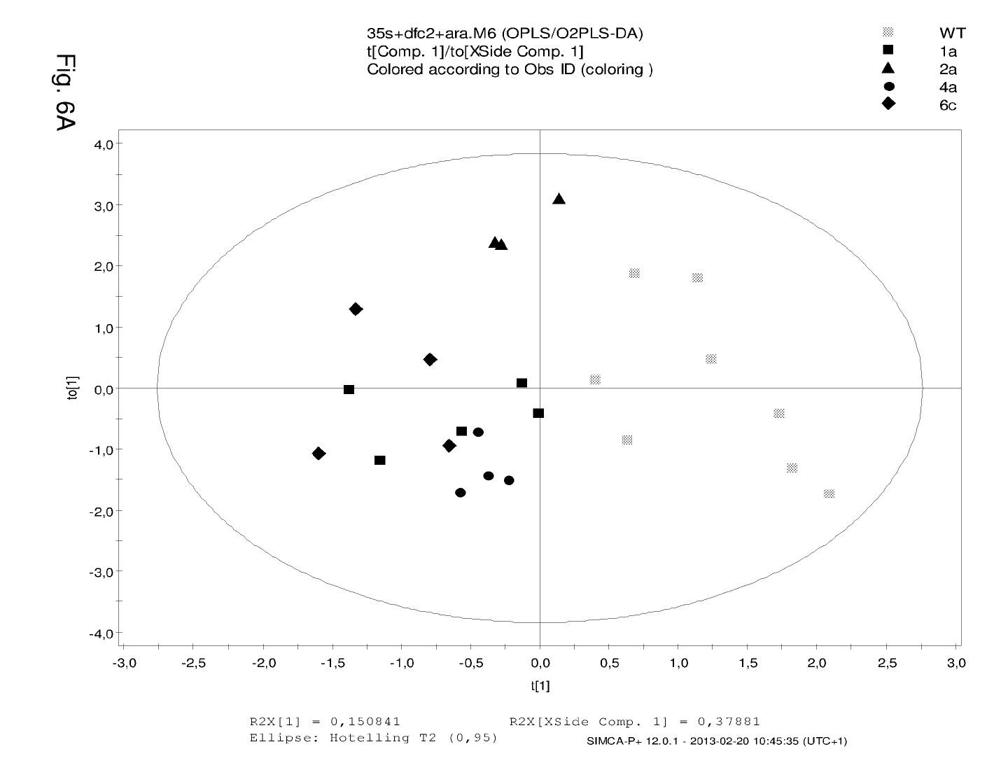

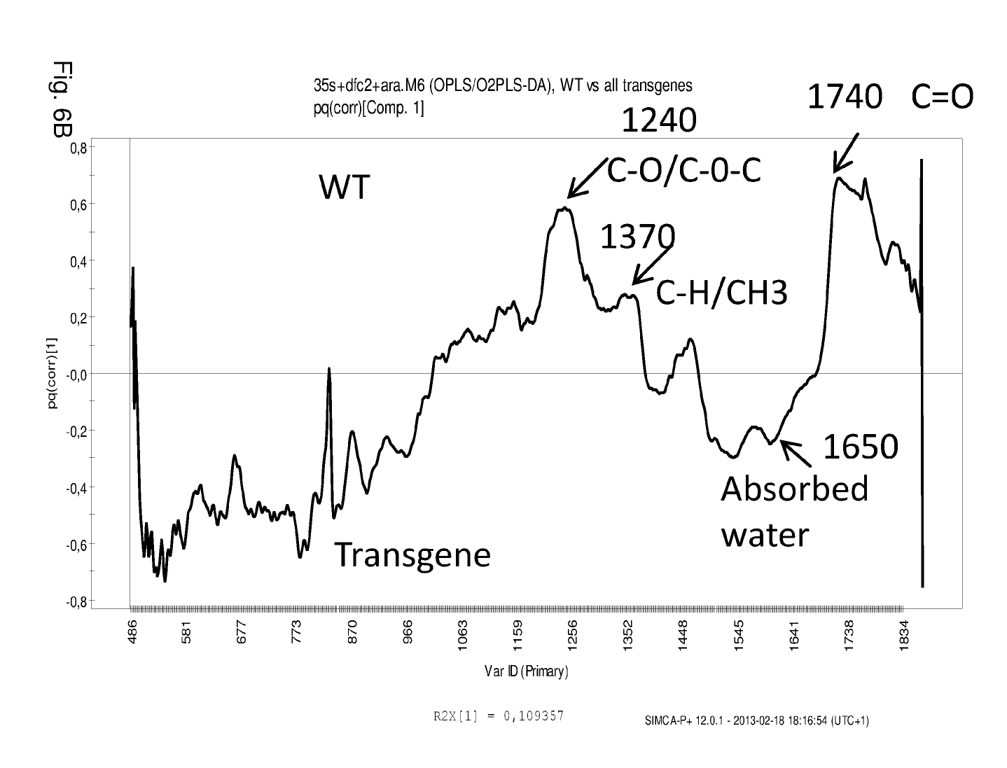

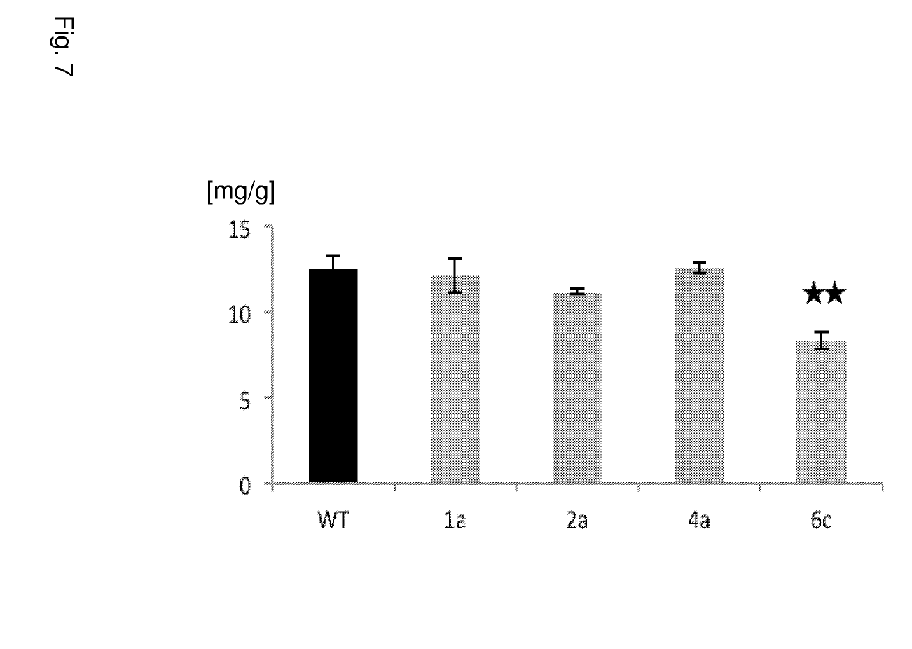

Since several xylan deficient mutants have irregular xylem phenotype, we analyzed xylem cell morphology in the transgenic lines and the WT, using xylem cell macerates (FIG. 5). No significant effects were observed on either fiber or vessel element size and shape.

Transgenic lines had altered stem chemistry as demonstrated by FT-IR analysis (FIG. 6). The spectra contributing to separation of WT from the transgenic lines included three wave numbers corresponding to acetic ester: 1240 cm⁻¹ corresponding to C—O and/or C—O—C, 1370 cm⁻¹ corresponding to CH3/CH bending, and 1730 cm⁻¹, corresponding to C═O. The signals at these wave numbers indicate that the there is less acetate in transgenic lines compared to the wildtype (WT) plants.

The total cell wall acetyl content in the stem was determined by analyzing release of acetic acid upon saponification with NaOH. The highly expressing line 6c showed 30% decrease in acetic acid release as compared to WT (FIG. 7).

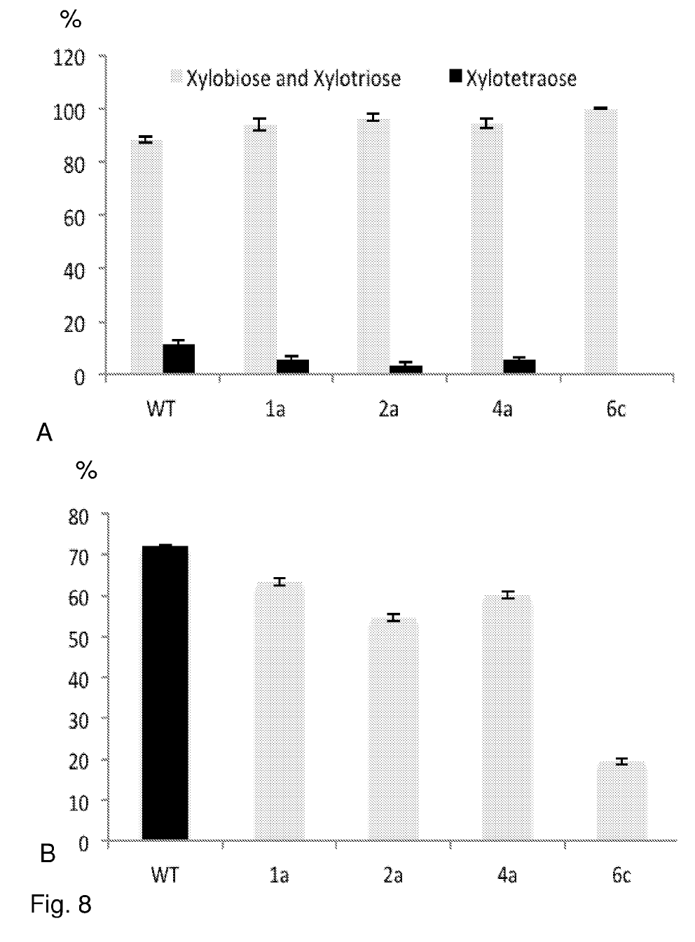

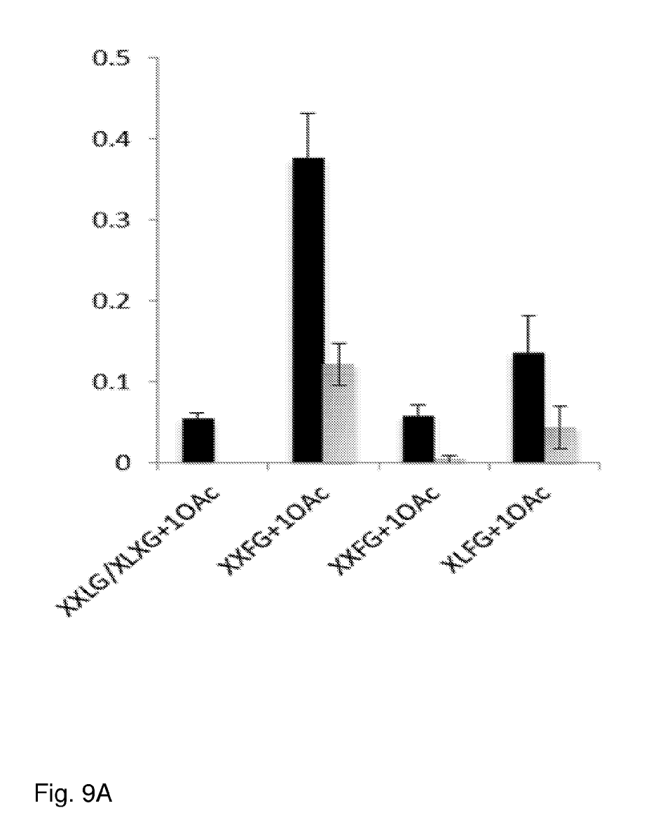

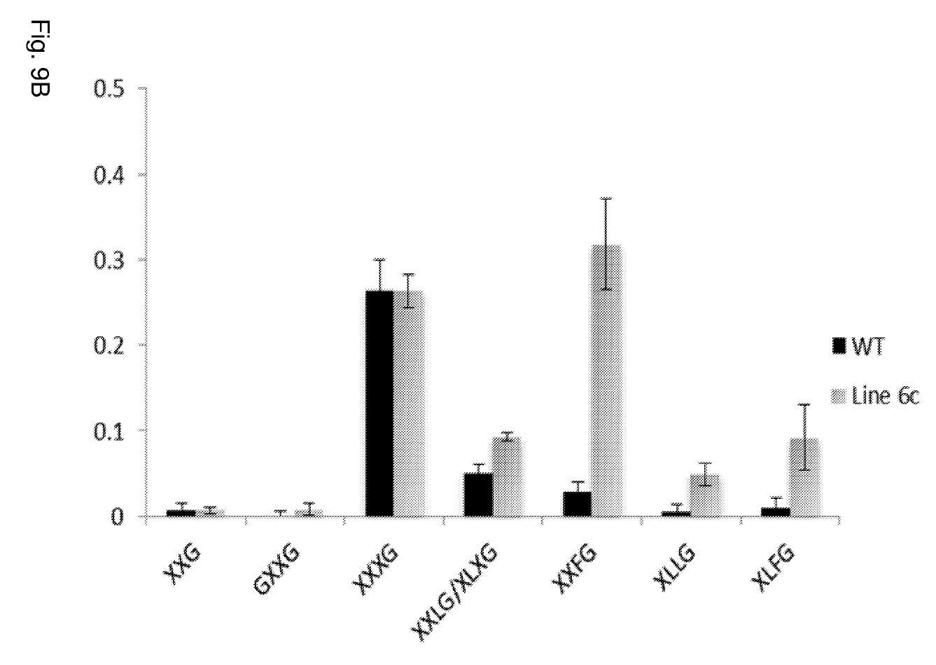

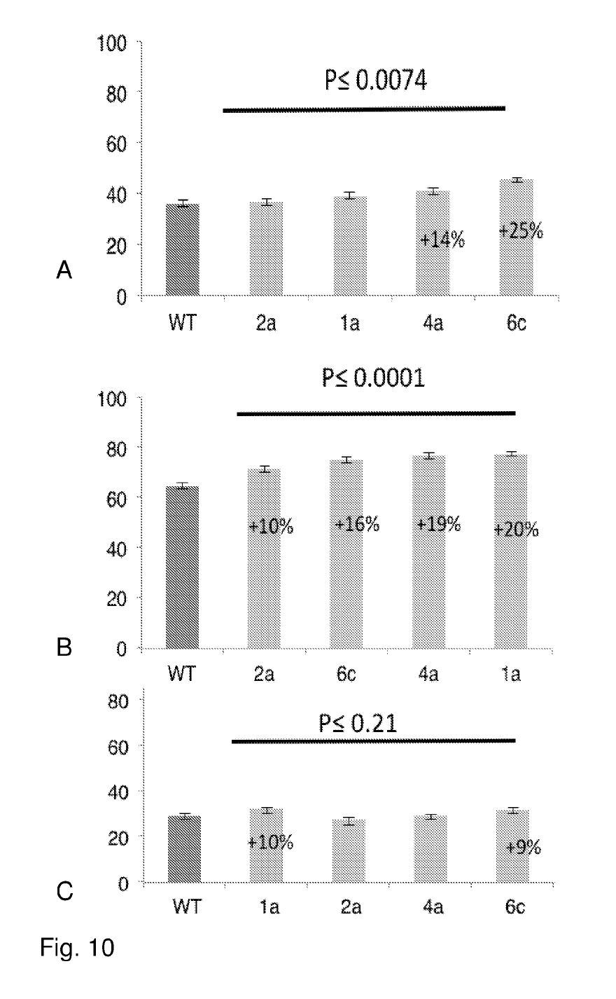

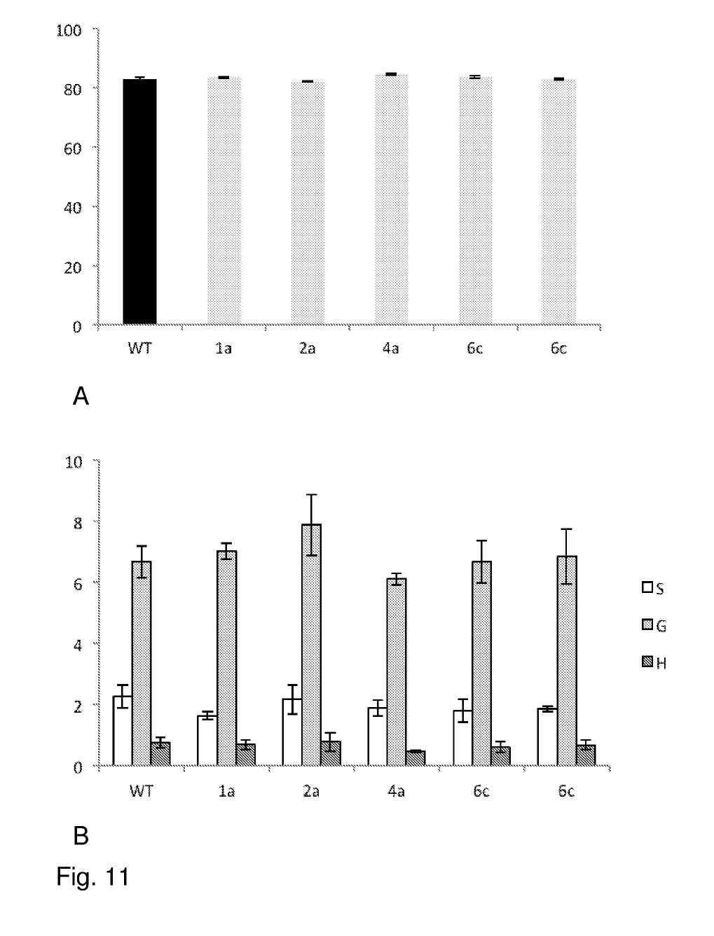

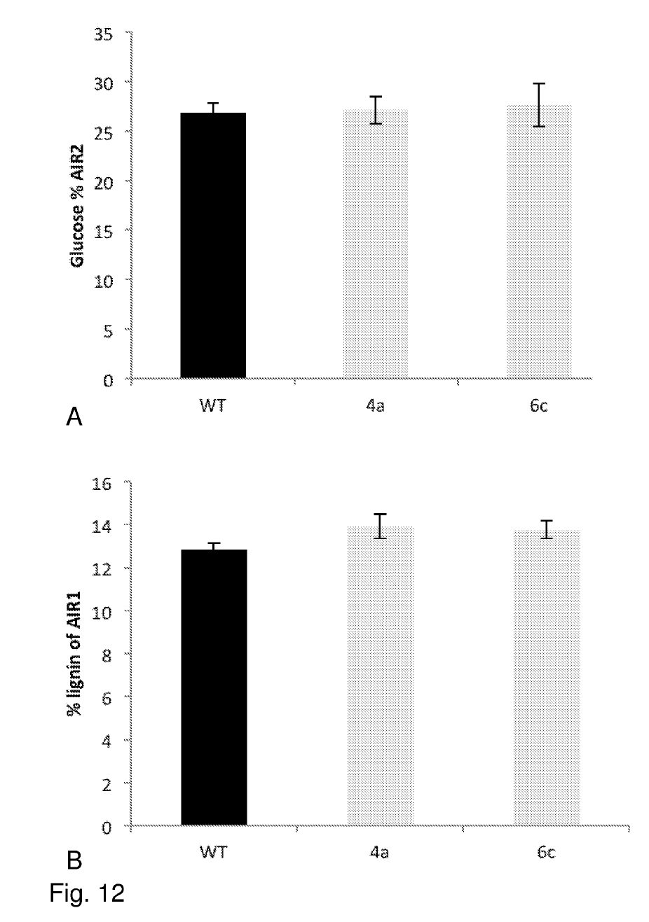

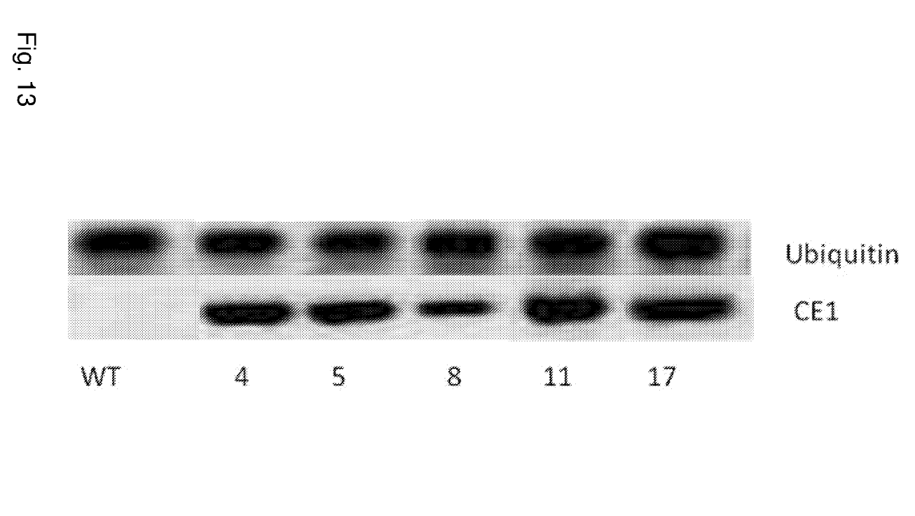

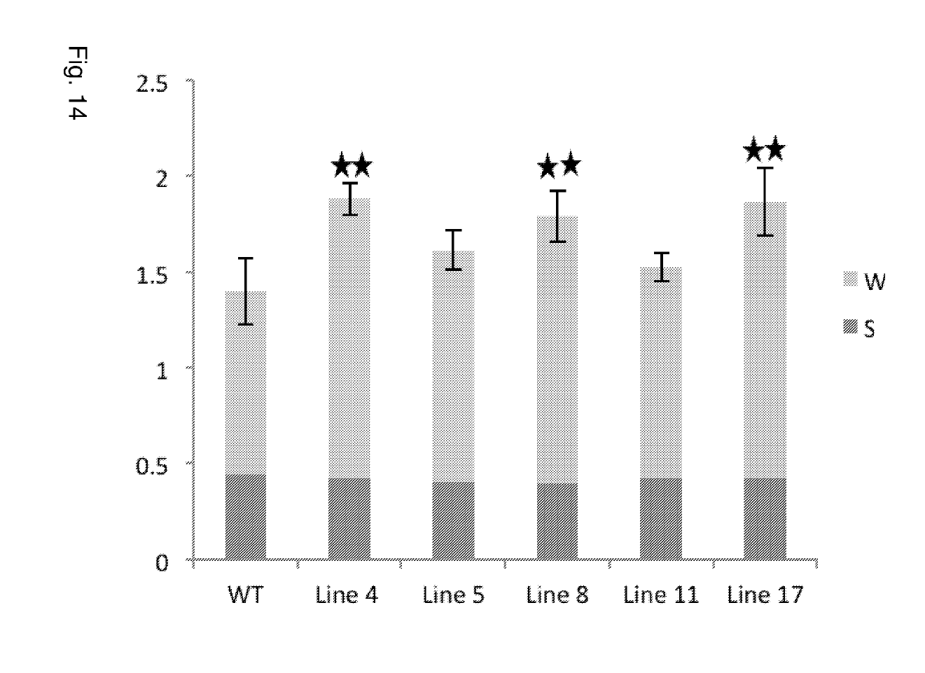

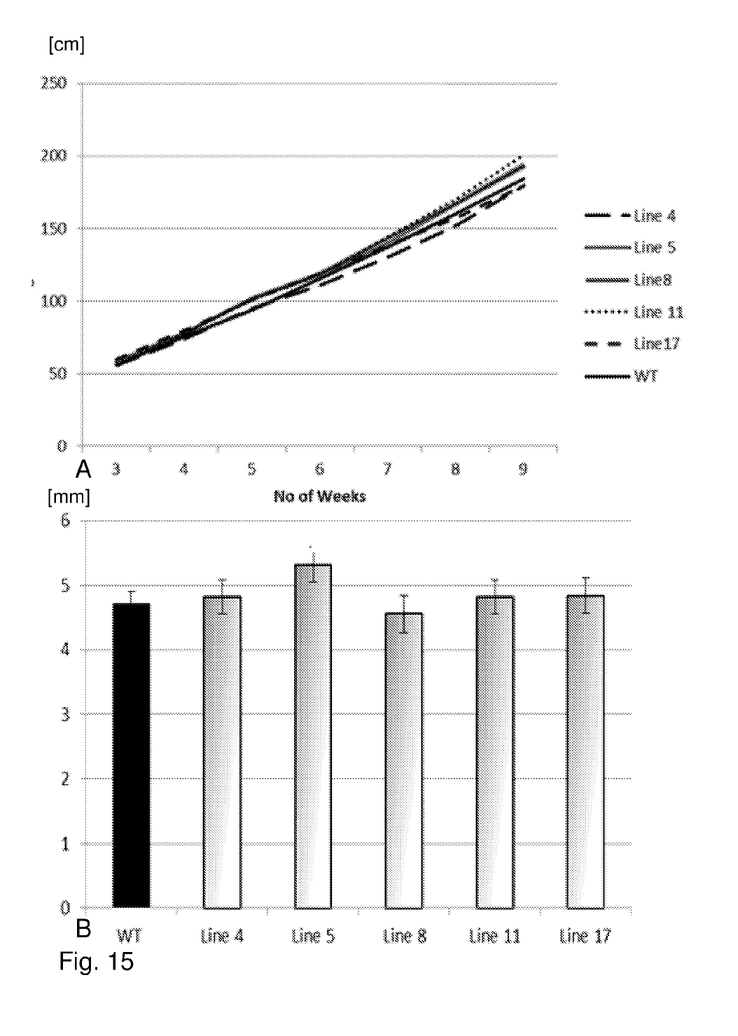

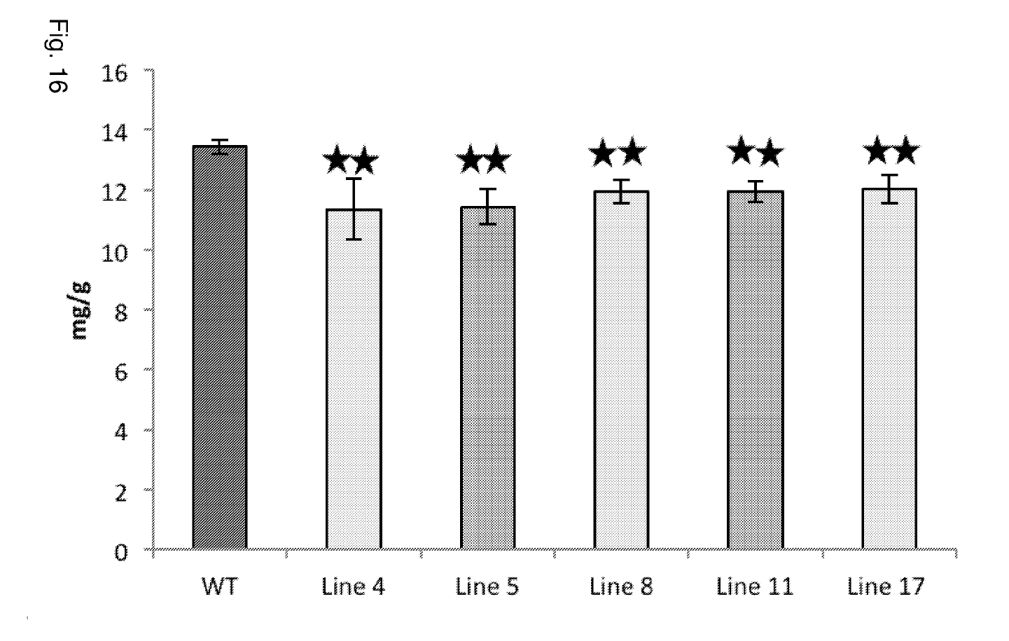

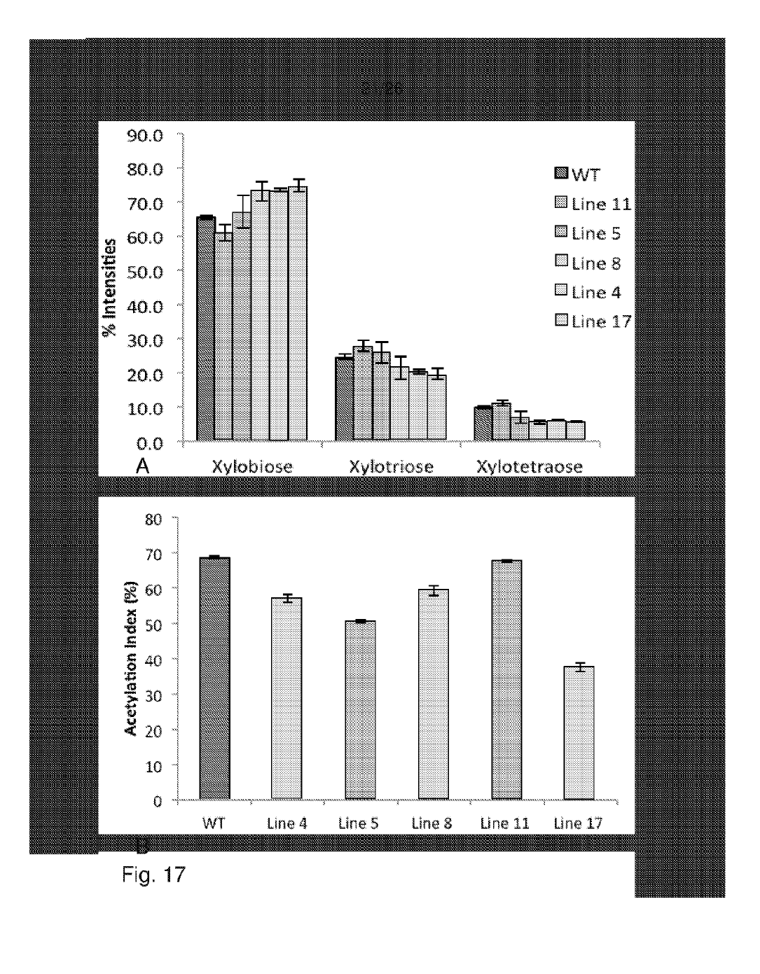

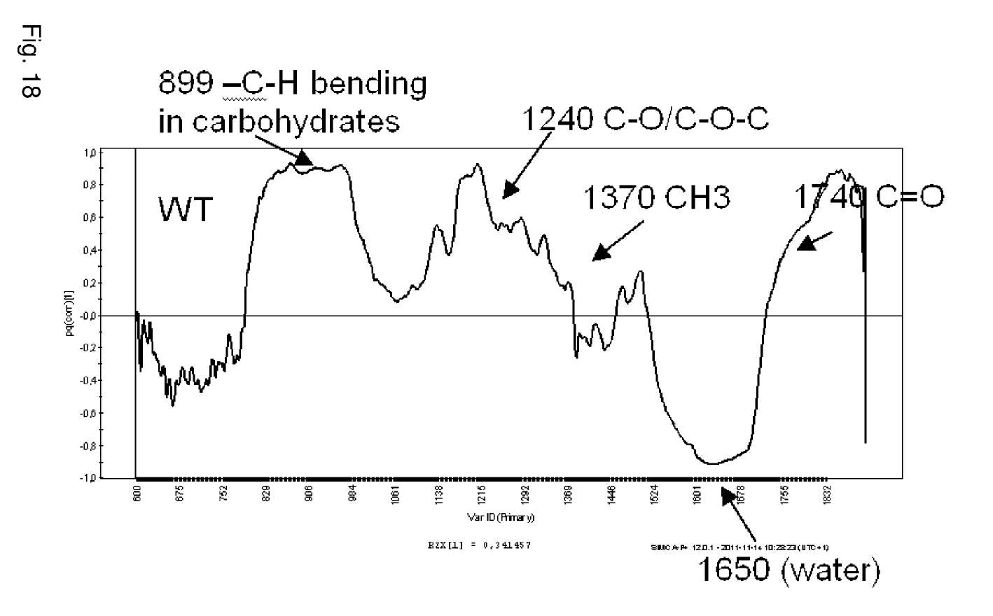

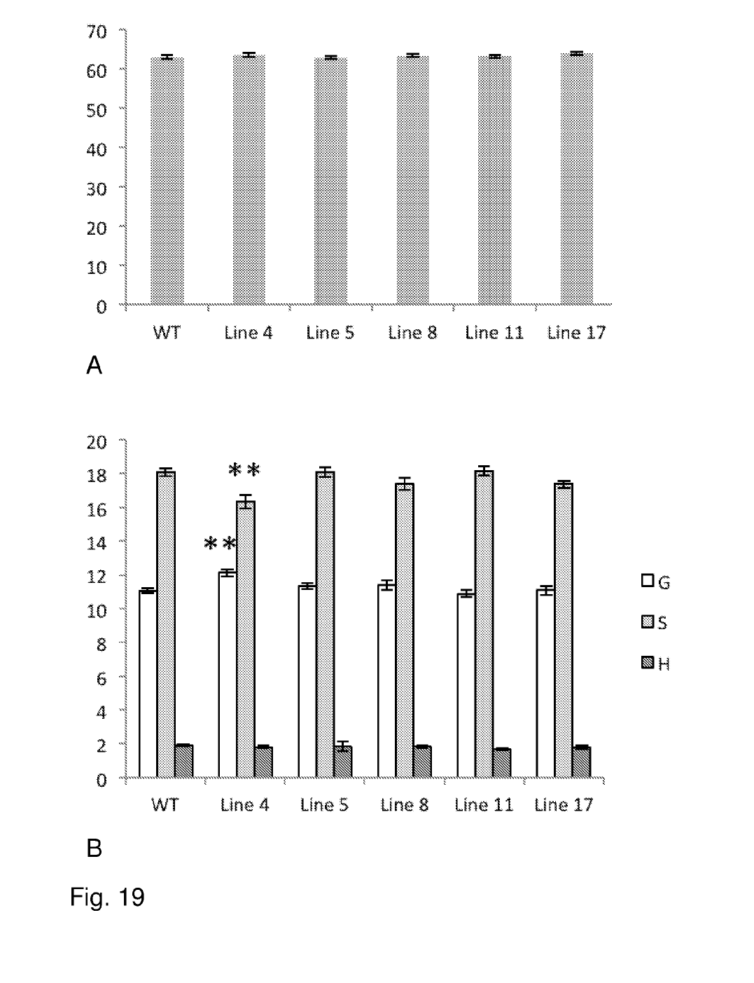

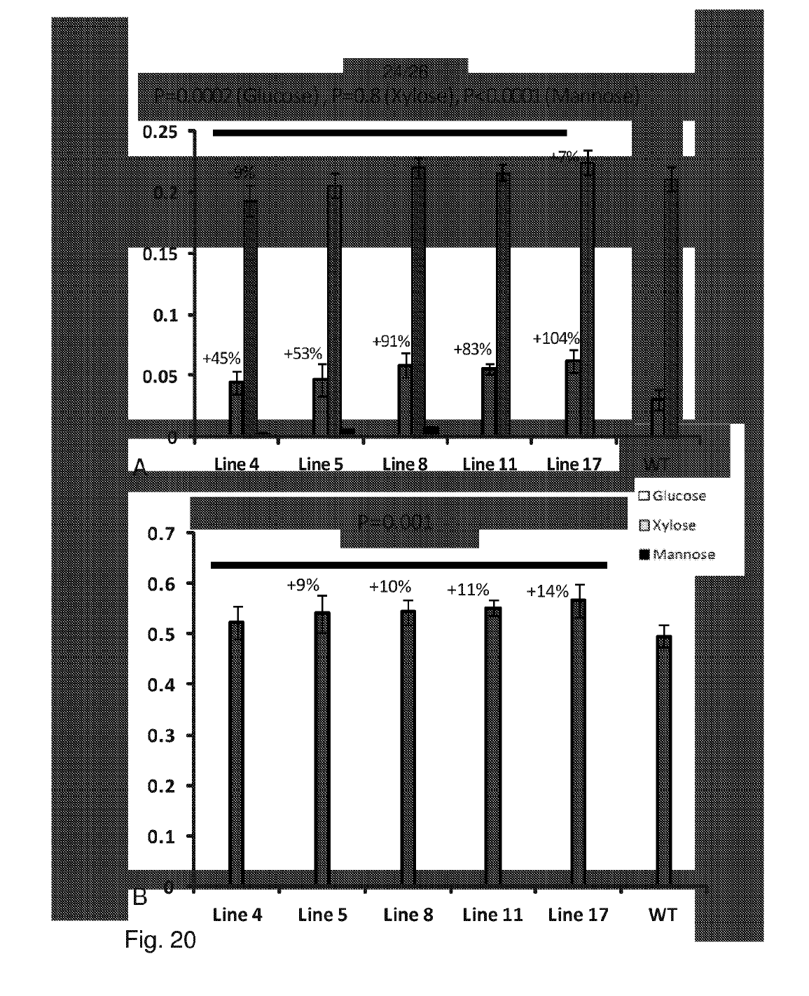

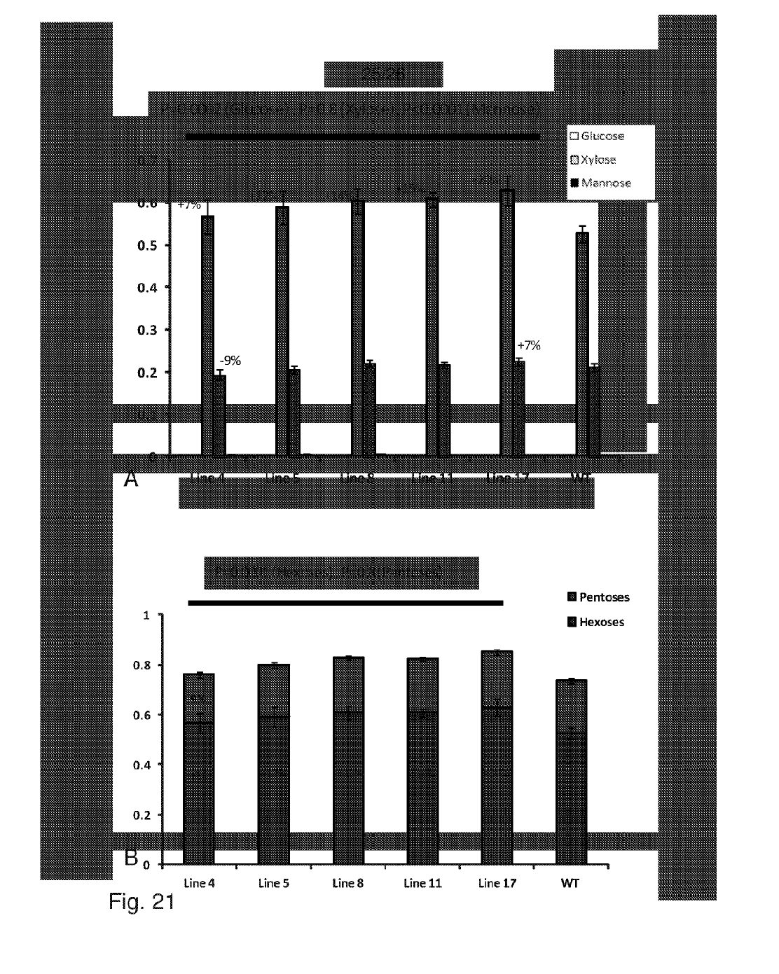

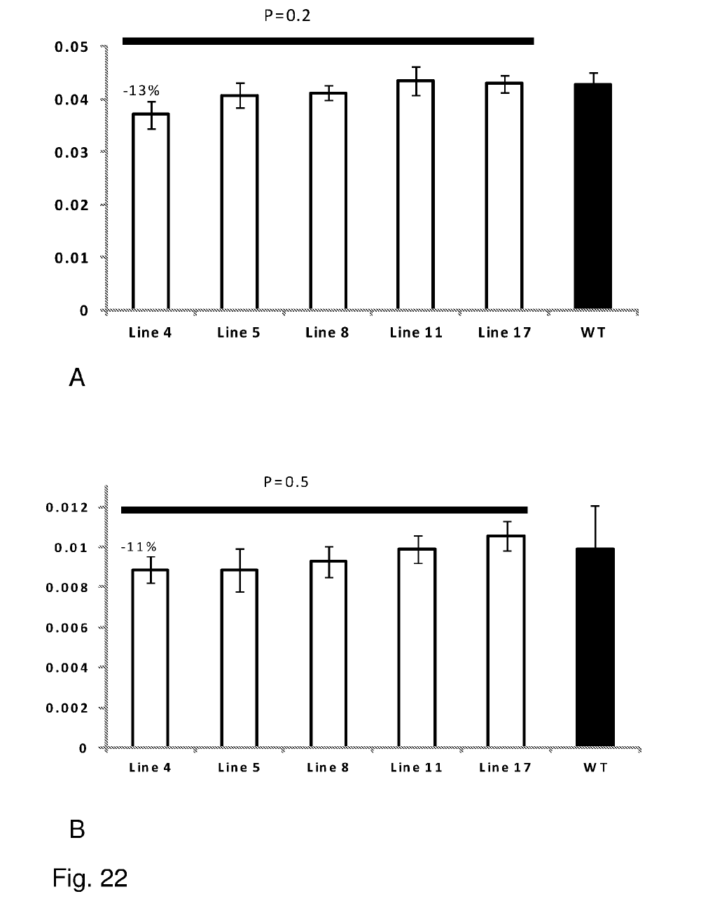

To examine the effects of the transgene on xylan acetylation, a MALDI-AP analysis of xylan oligosaccharides obtained from cell wall of transgenic lines and WT following xylanase hydrolysis was performed. FIG. 8 shows the different neutral oligosaccharides liberated by the xylanase. When the acetyl side chains are present, some xylan cannot be digested to xylobiose or xylotriose but instead, the xylotetraose is liberated as shown in FIG. 8A. Thus, the higher the acetylation, the more xylotetraose compared to xylobiose and xylotriose. According to this analysis, WT stem material had at least two times more xylotetraose than the weakest transgenic line (la). The strongest transgenic line 6c did not have any xylotetraose.

The different acetylated xylo-oligomers were detected and their total relative content relative to the total content of oligomers was calculated as acetylation index (FIG. 8B). This shows that the extent of xylan acetylation was reduced in transgenic lines, and the most affected line was line 6c.

To verify if the reduction of acetylation in plants overexpressing CE1 enzyme concerned also other polymers in addition to xylan, we analyzed oligosaccharide composition of cell wall material digested with a xyloglucan-specific glucanase by MALDI-TOF (FIG. 9). In xyloglucan, the acetyl ester groups are found on galacto-pyranose residue present in some of the side chains. The analysis showed that the WT contained more acetylated xylogluco-oligosaccharides containing galactose than line 6c. The result indicates that the reduction of acetyl content in the cell walls of the transgenic lines overexpressing CE1 enzyme concerns xyloglucan in addition to xylan. This result suggests a broad specificity of the CE1 enzyme used in transgenic lines.

Saccharification of stem lignocellulose of Arabidopsis was performed using three different types of pretreatment scenarios were applied: the chemical pretreatment with 0.5 M NaOH (Alkali pretreatment), the chemical pretreatment with 1% H₂SO₄ (Acid pretreatment), and no chemical pretreatment (water pretreatment) when the hot water was used only before the saccharification (FIG. 10). In the case of alkali and water pretreatment, the transgenic lines were releasing more sugar than the WT.

Production of ethanol by the fungus Trametes versicolor provided with lignocellulose prepared either from the plants of line 6c or from the WT plants. The fungus was digesting and fermenting the lignocellulose during the saccharification-fermentation cycle in liquid cultures over a period of several days. Ethanol was produced from both types of lignocellulose and it was detected in the medium after 5 days of culture. The ethanol yield was increased by 30%-50% when the lignocellulose from the line 6c was used compared to the production from the WT material. At the same time, the medium contained reduced level of acetic acid, a known inhibitor of fermentation and microorganism growth.

Stem chemical composition was analyzed by pyrolysis-GC. This analysis showed no statistically significant differences in carbohydrate or lignin contents between the transgenic lines and the WT (FIG. 11). These data were further confirmed by the Updegraff cellulose analysis and Klason lignin analysis in two selected lines, line 4a and 6c (FIG. 12). These data jointly indicate that the higher saccharification values in the transgenic lines are due to a higher sugar conversion i.e. more effective saccharification process of the material having similar carbohydrate and lignin contents.

In summary, the overexpression of fungal acetyl xylan esterase from family CE1 in Arabidopsis resulted in plants having reduced xylan acetylation. Acetyl was reduced in at least two matrix polymers in Arabidopsis: xylan and XG, suggesting a wide spectrum of action for the overexpressed enzyme. Consequently, higher saccharification was observed without chemical pretreatment as well as with alkali pretreatment. Thus the transgenic plants combined a higher ethanol production potential but with normal growth and normal cellulose and lignin content, and increased resistance to the biotrophic pathogen that was tested.

### Example 3

### Effects of CE1 Expression in Hybrid Aspen

Similar experiments with the same fungal construct were carried out in hybrid aspen (Populus tremula x tremuloides), clone T89. Transgenic lines with the presence of xylan esterase transcript were obtained (FIG. 13), multiplied and planted in the greenhouse. When we tested esterase activity in transgenic lines using substrate p-Napthyl Acetate as substrate, all the lines showed increase in enzyme activity as compared to WT and the increased activity was in the wall bound fraction (FIG. 14). Plant growth was mildly affected in some lines (FIG. 15). The height growth was increased in lines 5, 8 and 11, and decreased in line 4, without any relation to enzyme activity. Growth in diameter was not significantly affected.

The total cell wall acetyl content in the wood was decreased in all the transgenic lines as compared to WT down to 85% of the WT level in line 4 (FIG. 16).

Xylan acetylation was analyzed by MALDI-AP. Analysis shows that the acetylation level was reduced in xylan in all transgenic lines (FIG. 17).

Wood chemistry was further analyzed by FT-IR. The loading plots showing spectra separating the transgenic lines from the WT are shown in FIG. 18. The main differences were seen in the intensity of 899 cm⁻¹ band —C—H bending in hemicelluloses and cellulose, which was more abundant in the WT, and the intensity of 1650 cm⁻¹ corresponding to the adsorbed water, which was less abundant in the WT. Spectra corresponding to the acetyl group (1240, 1370 and 1740 cm⁻¹) were more intense in the WT, indicating a higher content of acetyl compared to the transgenic lines.

Pyrolysis-MS analysis of aspen wood did not reveal any change in carbohydrate spectra in transgenic lines (FIG. 19A). Spectra of S, G, and H lignin were unchanged except for line 4, which showed a small increase in G and decrease in S lignin compared to WT (FIG. 19B).

The saccharification of wood of aspen was investigated by using two different approaches: (1) enzymatic hydrolysis without pretreatment (FIG. 20A), and (2) acid pretreatment followed by enzymatic hydrolysis (FIG. 20B). Monosaccharide yields (arabinose, galactose, glucose, xylose and mannose) were determined using ion chromatography. The transgenic aspen lines showed improved glucose production rates and improved glucose yields compared to the wild-type (FIG. 20). Transgenic lines 4, 5 and 8 also showed significantly higher yields of mannose compared to the wild-type (FIGS. 21A).

The experiments with acid-pretreated aspen resulted in high yields of glucose and the transgenic lines had higher saccharification potential also under these conditions (FIGS. 20 and 21). Assuming a glucan content of around 55%, the theoretical maximum glucose yield would be around 0.61 g glucose per g wood (dry-weight), and yields at that level were feasible to achieve (FIG. 21). On average, the total yield of glucose of the transgenic lines was 13% higher than that of the WT (FIG. 21). From an industrial point of view it would be critical to achieve a high total sugar yield, and it is therefore noteworthy that the transgenic plants performed well in that type of experiments.

The yields of acetic acid without pretreatment and with acid pretreatment of transgenic lines and wild type aspen were investigated (FIG. 22). Line 4 showed significantly lower yield of acetic acid than the wild-type.

TABLE 1
The value in transgenic Arabidopsis lines expressed as % of the value in WT
(WT value = 100%).

| | | Sugar yield of | Sugar yield of | Sugar yield of |
|---|---|---|---|---|
| | | saccharification | saccharification | saccharification |
| | Acetylation | after water | after alkali | after acid |
| | compared to WT | pretreatment | pretreatment | pretreatment |
| WT Arabidopsis | % of WT | % of WT | % of WT | % of WT |
| Line1a | 97% | 108% | 120% | 110% |
| Line 2a | 89% | 102% | 110% | 94% |
| Line 4a | 100% | 114% | 119% | 99% |
| Line 6c | 66% | 125% | 116% | 109% |
| | From FIG. 7 | From FIG. 10A | From FIG. 10B | From FIG. 10C |

TABLE 2
The value in transgenic aspen lines expressed as % of the value in WT
(WT value = 100%).

| | | Glucose yield | Glucose yield | Yield of acetic | Yield of acetic acid in |
|---|---|---|---|---|---|
| | Acetylation | after acid | after | acid after acid | hydrolysates without |
| | compared to WT | pretreatment | hydrolysis | pretreatment | pretreatment |
| Aspen | % of WT | % of WT | % of WT | % of WT | % of WT |
| Line 4 | 85% | 145% | 105% | 87% | 89% |
| Line 5 | 85% | 153% | 109% | 95% | 89% |
| Line 8 | 89% | 191% | 110% | 96% | 94% |
| Line 11 | 89% | 183% | 111% | 102% | 100% |
| Line 17 | 89% | 204% | 114% | 101% | 107% |
| | From FIG. 16 | From FIG. 20A | From FIG. 20B | From FIG. 22A | From FIG. 22B |

In summary, it has been shown that it is possible to decrease acetylation of xylan in plants without compromising their growth and development. No major effects on cell wall composition, except for changes in acetyl content, were shown in Arabidopsis and aspen transgenic plants. Major improvement in saccharification of aspen wood was observed without pretreatment and with dilute acid pretreatment (up to >40%), with changes in different monosaccharide released and minor change in acetic acid release.

1. A method for increasing saccharification potential in a plant, said method comprising overexpressing a polynucleotide encoding an acetyl xylan esterase polypeptide in at least one cell type in said plant.

2. The method according to claim 1, further comprising increasing glucose yields in the said plant.

3. A method for producing a genetically modified plant, comprising overexpressing a polynucleotide encoding an acetyl xylan esterase polypeptide in at least one cell type in said plant.

4. The method according to claim 3, wherein the said plant has increased saccharification potential as compared to a corresponding non-genetically modified wild-type plant.

5. The method according to claim 1, said method comprising transforming said cell type with an expression cassette comprising a promoter that is functional in a plant cell, said promoter being operably linked to a polynucleotide encoding an acetyl xylan esterase polypeptide, and said promoter regulating overexpression.

6. The method according to claim 5, wherein the said promoter is a CaMV 35S promoter.

7. The method according to claim 1, wherein the said polynucleotide is selected from:
(a) polynucleotides comprising the nucleotide sequence of SEQ ID NO: 1;
(b) polynucleotides comprising a nucleotide sequence capable of hybridizing, under stringent hybridization conditions, to a nucleotide sequence complementary the polypeptide coding region of a polynucleotide as defined in (a) and which codes for a biologically active acetyl xylan esterase polypeptide or a functionally equivalent modified form thereof; and
(c) polynucleotides comprising a nucleic acid sequence which is degenerate as a result of the genetic code to a nucleotide sequence as defined in (a) or (b) and which codes for a biologically active acetyl xylan esterase polypeptide or a functionally equivalent modified form thereof.

8. The method according to claim 1, wherein the said acetyl xylan esterase polypeptide is selected from:
(a) polypeptides comprising the amino acid sequence shown as SEQ ID NO: 2, 3, 4 or 5;
(b) polypeptides consisting essentially of the amino acid sequence shown as SEQ ID NO: 2, 3, 4 or 5; and
(c) polypeptides consisting of the amino acid sequence shown as SEQ ID NO: 2, 3, 4 or 5.

9. The method according to claim 1, wherein the plant is selected from angiosperms and other plants having acetylated xylan in their cell walls.

10. The method according to claim 9, wherein the plant is of the family Salicaceae.

11. The method according to claim 1, wherein the plant or a part of the plants is pretreated with a suitable agent, such as acid or alkali, prior to enzymatic hydrolysis.

12. The method according to claim 1 for increasing resistance to pathogens in the said plant.

13. A genetically modified plant produced by the method according to claim 3.

14. A genetically modified plant overexpressing a polynucleotide encoding an acetyl xylan esterase polypeptide in at least one cell type in said plant.

15. The genetically modified plant according to claim 14, wherein the said plant has increased saccharification potential as compared to a corresponding non-genetically modified wild-type plant.

16. The genetically modified plant according to claim 13, wherein the said plant has increased glucose yields as compared to a corresponding non-genetically modified wild-type plant.
